(19) United States
(12) Patent Application Publication
(10) Pub. No.: US 2025/0277068 A1
(43) Pub. Date: Sep. 4, 2025

(54) GASIFICATION PROCESS

(71) Applicant: Bridgestone Americas Tire Operations, LLC, Nashville, TN (US)

(72) Inventors: Robert W. WHITE, Gilbert, AZ (US); Terrence E. HOGAN, Uniontown, OH (US)

(21) Appl. No.: 19/068,337

(22) Filed: Mar. 3, 2025

Related U.S. Application Data
(60) Provisional application No. 63/560,070, filed on Mar. 1, 2024.

(51) Int. Cl. C08F 36/06; C10J 3/46

(52) U.S. Cl. CPC C08F 36/06; C10J 3/46; C10J 2300/0946

(57) ABSTRACT

A gasification process comprising (i) providing a carbonaceous feedstock; and (ii) gasifying the carbonaceous feedstock in the presence of a catalyst.

This application claims the benefit of U.S. Provisional Application No. 63/560,070 filed on Mar. 1, 2024, which is incorporated herein by reference.

### FIELD OF THE INVENTION

Embodiments of the present invention provide a method for gasifying carbonaceous feedstocks in the presence of a catalyst to produce syngas product streams desirable for bioconversion to useful products.

### BACKGROUND OF THE INVENTION

With the goal of carbon circularity, techniques have been proposed whereby carbon monoxide and hydrogen are biosynthetically reacted to produce useful hydrocarbon-based molecules such as alcohols. These molecules can then be used as feedstock for the synthesis of a variety of useful molecules including polymers. Carbon monoxide and hydrogen have historically been produced in the production of syngas. For example, steam methane reforming techniques react water and methane in the gaseous phase to produce mixtures of carbon monoxide and hydrogen, which is known as synthetic gas or syngas. Syngas is also conventionally produced by gasification techniques whereby carbonaceous materials, such as biomass or municipal solid waste, are thermally decomposed in the presence of limited amounts of oxygen and/or water to produce gaseous streams that can be rich in carbon monoxide and hydrogen. While gasification is a desirable technique for consuming biomass and municipal solid waste, the resulting gaseous stream can also include significant levels of carbon dioxide, methane, and nitrogen. When the resulting gaseous stream (i.e. syngas) is biosynthetically converted to other molecules, these other constituents (e.g. methane and nitrogen) are not consumed within the biosynthetic reaction and therefore must be considered and/or managed within the product stream. And, carbon dioxide in the syngas requires additional hydrogen to biosynthetically convert the carbon dioxide. Since hydrogen is already a limiting factor, the presence of carbon dioxide in the product stream reduces the efficiency of the overall process.

### SUMMARY OF THE INVENTION

One or more embodiments of the present invention provide a gasification process comprising (i) providing a carbonaceous feedstock; and (ii) gasifying the carbonaceous feedstock in the presence of a catalyst.

### DETAILED DESCRIPTION OF ILLUSTRATIVE EMBODIMENTS

Embodiments of the invention are based, at least in part, on the discovery of a process for the gasification of carbonaceous feedstock to produce gaseous streams that include advantageous levels of carbon monoxide relative to other carbon molecules in the product stream such as methane and carbon dioxide. Also, embodiments of the invention provide processes that produce gasification product streams with advantageous levels of hydrogen. The gasification product streams of this invention can advantageously be directed toward biosynthetic processes that consume carbon monoxide and hydrogen to produce useful hydrocarbon-based molecules, such as alcohols, which may be useful for the synthesis of other desirable molecules such as monomers (e.g. butadiene), which can ultimately be converted to polymers. According to embodiments of the invention, gasification of the carbonaceous feedstock takes place in the presence of a catalyst that promotes the production of carbon monoxide and optionally hydrogen. In particular embodiments, the feedstock includes used tires, and it is contemplated that the techniques proposed by the present invention will yield useful gasification product streams. Also, it is contemplated that practice of the present invention will improve overall carbon efficiency since gasification of the carbonaceous feedstock can take place at lower temperatures.

### Carbonaceous Feedstock

In one or more embodiments, the carbonaceous feedstock, which may simply be referred to as feedstock, may include tire feedstock from used tires, which may also be referred to as used tire feedstock, tire feedstock, or used tires. As the skilled person appreciates, tire feedstock may include vulcanized polymer, carbon black filler, silica, resins, oils, fibrous yarn, and metal. The vulcanized polymer may include the sulfur-crosslinked residue of natural rubber and/or one or more synthetic elastomers including diene polymers and copolymers. In one or more embodiments, the used tire feedstock may include shredded or otherwise ground tires with one or more constituents of the used tire removed. For example, the tire feedstock may be treated to remove metal by methods known in the art (e.g. magnetic separation). Alternatively, or in combination therewith, the used tire feedstock may be optionally treated to remove fibrous reinforcement such as fiber yarn or cord, which the skilled person understands is often found in conjunction with the vulcanized rubber within many tire components. Alternatively, or in combination with the foregoing, the used tire feedstock may be optionally treated to remove inorganic materials such as silica filler, which the skilled person appreciates is often found used tire components. In any event, the tire feedstock can be processed into tire shreds, tire chips, or ground or crumb rubber and fed to the thermal decomposition unit.

In one or more embodiments, the tire feedstock is characterized by relatively low amounts of metal, which may result from pre-treatment of the tire feedstock to remove metal. In one or more embodiments, the tire feedstock includes less than 25 wt %, in other embodiments less than 15 wt %, and in other embodiments less than 1 wt % metal based on the entire weight of the feedstock fed to thermal decomposition in accordance with the present invention.

In one or more embodiments, the tire feedstock is characterized by relatively low amounts of fibrous yarn or cord, which may result from pre-treatment of the tire feedstock to remove fibrous yarn or cord. In one or more embodiments, the tire feedstock includes less than 5 wt %, in other embodiments less than 4 wt %, in other embodiments less than 3 wt %, in other embodiments less than 2 wt %, and in other embodiments less than 1 wt % fibrous yarn or cord based on the entire weight of the feedstock fed to thermal decomposition in accordance with the present invention.

In one or more embodiments, the tire feedstock is characterized by relatively low amounts of inorganic filler (e.g. silica), which may result from pre-treatment of the tire feedstock to remove inorganic filler. In one or more embodiments, the tire feedstock includes less than 30 wt %, in other embodiments less than 20 wt %, in other embodiments less than 10 wt %, and in other embodiments less than 5 wt % inorganic filler based on the entire weight of the feedstock fed to thermal decomposition in accordance with the present invention.

In one or more embodiments, the used tire feedstock includes tire remains from passenger tires. In other embodiments, the used tire feedstock includes tire remains from non-passenger tires such as, but not limited to, truck and bus tires, off-road vehicle tires, agricultural tires, and race tires.

In one or more embodiments, the used tires may be characterized by a compacted density of greater than 640 kg/m³, in other embodiments greater than 720 kg/m³, and in other embodiments greater than 770 kg/m³, where density is determined by ASTM D 698-07.

In one or more embodiments, the carbonaceous feedstock includes carbonaceous materials other than the tire feed stock, which may also be referred to as non-tire feedstock. Carbonaceous material refers to any carbon material whether in solid, liquid, gas, or plasma state. Non-limiting examples of carbonaceous materials that can be used as feedstock include carbonaceous liquid product, industrial liquid recycle, municipal solid waste (MSW or msw), urban waste, agricultural material, forestry material, wood waste, construction material, vegetative material, industrial waste, fermentation waste, petrochemical coproducts, alcohol production coproducts, coal, plastics, waste plastic, coke oven tar, lignin, black liquor, polymers, waste polymers, polyethylene terephthalate (PETA), polystyrene (PS), sewage sludge, animal waste, crop residues, energy crops, forest processing residues, wood processing residues, livestock wastes, poultry wastes, food processing residues, ethanol coproducts, spent grain, spent microorganisms, municipal waste, construction waste, demolition waste, biomedical waste, hazardous waste, or their combinations. In one or more embodiments, the carbonaceous material includes biomass. In one or more embodiments, the biomass is bagasse including, but not limited to, the bagasse of sugar cane, sorghum, and guayule plant.

In one or more embodiments, the carbonaceous feedstock includes guayule bagasse, which is produced as the result of a process to extract rubber and resin from the guayule plant. These processes are known in the art including those described in U.S. Publication No. 2022/0356273, which is incorporated herein by reference. Methods for the desolventization of guayule bagasse are described in U.S. Pat. No. 10,132,563, which is also incorporated herein by reference. In one or more embodiments, the guayule bagasse contains no more than 1 wt % organic solvent (based upon the total weight of the dried bagasse). In certain embodiments, the dried bagasse contains no more than 0.5 wt % organic solvent (based upon the total weight of the dried bagasse). In one or more embodiments, the dried bagasse may contain a quantity of water and higher boiling point terpenes. In certain embodiments, the total quantity of water and higher boiling point terpenes in the dried bagasse may be higher than the content of organic solvents. In certain embodiments, resin content (including the higher boiling point terpenes) in the dried bagasse is generally acceptable and, in some instances, useful.

In one or more embodiments, the non-tire feedstock (e.g. biomass or municipal waste) may be characterized by a compacted density of less than 600 kg/m³, in other embodiments less than 580 kg/m³, and in other embodiments less than 560 kg/m³, where density is determined by ASTM D 698-07.

In one or more embodiments, the carbonaceous feedstock includes a combination of tire feedstock and non-tire feedstock. In one or more embodiments, the carbonaceous feedstock includes from about 0 to about 95, in other embodiments from about 1 to about 75, and in other embodiments from about 2 to about 55 wt % non-tire feedstock with the balance including used tire. In one or more embodiments, the feedstock includes less than 95, in other embodiments less than 80, and in other embodiments less than 70 wt % non-tire feedstock with the balance including used tires. In these or other embodiments, the feedstock includes greater than 10, in other embodiments greater than 20, in other embodiments greater than 30, in other embodiments greater than 40, in other embodiments greater than 50, and in other embodiments greater than 70 wt % used tires, with the balance including non-tire feedstock.

In alternative embodiments, the feedstock is substantially, and in certain embodiments exclusively, comprised of non-tire feedstock. For example, in one or more embodiments, the feedstock includes greater than 80 wt %, in other embodiments greater than 90 wt %, and in other embodiments greater than 99 wt % municipal solid waste. Or, in other exemplary embodiments, the feedstock includes greater than 80 wt %, in other embodiments greater than 90 wt %, and in other embodiments greater than 99 wt % biomass.

### Gasification Reaction

In one or more embodiments, the gasification reaction takes place a temperature of greater than 500° C., in other embodiments greater than 600° C., in other embodiments greater than 700° C., in other embodiments greater than 800° C., in other embodiments greater than 900° C., and in other embodiments greater than 1000° C., in other embodiments greater than 1200° C., in other embodiments greater than 1400° C., in other embodiments greater than 1600° C., and in other embodiments greater than 1800° C. In these or other embodiments, the gasification reaction takes place at a temperature of less than 2500° C., in other embodiments less than 2300° C., in other embodiments less than 2100° C., in other embodiments less than 1900° C., in other embodiments less than 1800° C., in other embodiments less than 1600° C., and in other embodiments less than 1400° C. In one or more embodiments, the gasification reaction takes place at temperatures of from about 500 to about 2500° C., in other embodiments from about 800 to about 2300° C., in other embodiments from about 1000 to about 2000° C., in other embodiments from about 1100 to about 1800° C., and in other embodiments from about 1200 to about 1500° C. In one or more embodiments, the gasification reaction takes place at a temperature above the fusion temperature of the char. While it has been observed that higher levels of char are produced at lower temperatures, it is contemplated that practice of the present invention will allow the gasification reaction to proceed at lower temperatures, which is believed to be advantageous since high temperatures, which require higher oxygen levels, result in higher levels of carbon dioxide in the product stream.

In one or more embodiments, the gasification reaction takes place in the presence of thresholds levels of oxygen. The skilled person appreciates that the amount of oxygen present during gasification can be quantified based upon the oxygen equivalence ratio (λ), which is defined as the ratio between the supplied O₂ and the stoichiometric O₂ demand for complete combustion:

$$λ = \frac{{\dot{m}}_{actual}}{{\dot{m}}_{stoichiometric}}$$

where \dot{m}_{actual} and \dot{m}_{stoichiometric} are the actually supplied O₂ mass flow (kg/s) and the stoichiometrically required (for complete combustion) O₂ mass flow (kg/s), respectively. In one or more embodiments, the gasification reaction takes place in the presence of an amount of oxygen to provide an oxygen equivalence ratio of greater than 0.05, in other embodiments greater than 0.1, and in other embodiments greater than 0.2. In these or other embodiments, the gasification reaction takes place in the presence of an amount of oxygen to provide an oxygen equivalence ratio less than 0.8, in other embodiments less than 0.6, and in other embodiments less than 0.5. In one or more embodiments, the gasification reaction takes place in the presence of an amount of oxygen to provide with an oxygen equivalence ratio of from about 0.05 to about 0.8, in other embodiments from about 0.1 to about 0.6, and in other embodiments from about 0.2 to about 0.5.

In one or more embodiments, any required oxygen may be supplied as air, oxygen mixed with nitrogen, or as pure oxygen. In one or more embodiments, the purity of the oxygen delivered to the gasification reaction is greater than 50 vol. percent, in other embodiments greater than 80 vol. percent, in other embodiments greater than 90 vol. percent, and in other embodiments greater than 95 vol. percent.

The gasification furnaces, which may also be referred to as gasifiers, that may be employed in the practice of the present invention include those that have been commercially used including, but not limited to, counter-current fixed bed gasifiers, co-current fixed bed gasifiers, fluidized bed gasifiers, entrained flow gasifiers, plasma gasifiers, and free radical gasifiers. In one or more embodiments, a multi-zone gasification furnace is employed. Useful gasifiers are disclosed in U.S. Publ. Nos. 2020/0078728, 2021/0340446, 2016/0053992, and 2017/0247618, which are incorporated herein by reference. Useful gasification furnaces and processes are commercially available including those available from Sierra Energy.

### Catalyst

As indicated above, the gasification reaction takes place in the presence of a catalyst that promotes the formation of carbon monoxide and optionally promotes the liberation of hydrogen. In one or more embodiments, the catalyst is an alkali metal or alkali metal salt. In these or other embodiments, the catalyst is an alkaline earth metal or alkaline earth salt. In these or other embodiments, the catalyst is a transition metal or transition metal salt. For example, the alkali metal may include potassium, lithium and/or sodium. The alkaline earth metal may include, for example, barium, magnesium and/or calcium. And, the transition metals may include, for example, bismuth, antimony, boron, iron, chromium, zirconium, lead, copper, aluminum, nickel, titanium, cobalt, vanadium, manganese, zinc, and molybdenum. As the skilled person appreciates, salts can be formed as oxide, carbonates, sulfates, and halides. Specific examples of useful catalysts include potassium carbonate, lithium carbonate, sodium chloride, Raney nickel (inactivated), Raney nickel (activated with sodium hydroxide), nickel (II) chloride hydrate, lead (II) oxide, zinc oxide, lead (IV) oxide, strontium (I) oxide, boron trioxide, copper (II) oxide, copper (I) oxide, barium oxide, antimony (III) oxide, molybdenum (III) oxide, cobalt (I) oxide, bismuth (III) oxide, zinc (II) bromide, magnesium oxide, titanium (II) oxide, tin (II) oxide, iron (IV) oxide, nickel (III) oxide, nickel (I) oxide, aluminum (III) oxide, chromium (III) oxide, manganese (II) oxide, zirconium (II) oxide, calcium (II) hydroxide, and iron (0).

According to aspects of the invention, the catalyst is applied to the carbonaceous feedstock in particulate form. In one more embodiments, the catalyst particles are characterized by a particle size (D90) of greater than 5 μm, in other embodiments greater than 25 μm, in other embodiments greater than 50 μm, and in other embodiments greater than 100 μm. In these or other embodiments, the catalyst particles are characterized by a particle size (D90) of less than 5 mm, in other embodiments less than 2.5 mm, in other embodiments less than 1 mm, and in other embodiments less than 0.1 mm. In one or more embodiments, the catalyst particles are characterized by a particle size (D90) of from about 5 μm to about 5 mm, in other embodiments from about 50 μm to about 1 mm, and in other embodiments from about 100 μm to about 0.1 mm. According to these embodiments, the particulate can be applied to the carbonaceous feedstock employing conventional particle sprinkling or distribution devices.

In other embodiments, the catalyst is applied to the carbonaceous feedstock in the form of an emulsion or dispersion such as an emulsion in water. For example, the emulsion or dispersion can be sprayed on to the feedstock prior to delivery of the feedstock to the gasifier. Or, in other embodiments, the emulsion or dispersion can be sprayed on to the feedstock within the gasifier.

Aspects of the invention concern the catalyst loading relative to the weight of the feedstock. In one or more embodiments, the process includes introducing greater than 0.01, in other embodiments greater than 0.05, in other embodiment greater than 0.1, in other embodiments greater than 0.5, and in other embodiments greater than 4 wt % catalyst based upon the weight of the feedstock. In these or other embodiments, the process includes introducing less than 20, in other embodiments less than 10, in other embodiments less than 7, and in other embodiments less than 6 wt % catalyst based upon the weight of the feedstock. In one or more embodiments, the process includes introducing from about 0.01 to about 20, in other embodiments from about 0.1 to about 10, in other embodiments from about 0.5 to about 7, and in other embodiments from about 4 to about 6 wt % catalyst based upon the weight of the feedstock.

### Catalyst Recovery

As the skilled person appreciates, the primary product stream from a gasifier is the gaseous product stream that includes carbon dioxide, hydrogen, carbon dioxide, methane, and other optional constituents. The by-product stream from the gasifier is a solid that is conventionally referred to as the gasifier bottoms. These bottoms, which are also referred to as char or glass, typically include non-volatilized carbonaceous, inorganic materials, and metals. It is contemplated that the catalyst introduced to the feedstock and/or gasifier, either in its original form or as a residue, will transfer to the gaseous product stream or the char.

In one or more embodiments, the process of the present invention includes recovering the catalyst compounds and/or catalyst metals from the gaseous product stream and/or the char. The recovered catalyst compounds and/or catalyst metals can be reused in subsequent gasification reactions to form a continuous recycling operation. According to one or more embodiments of the invention, greater than 30 wt %, in other embodiments greater than 40 wt %, in other embodiments greater than 50 wt %, in other embodiments greater than 60 wt %, and in other embodiments greater than 65 wt % of the catalyst introduced to the gasification reaction is subsequently recovered and returned to the gasifier for subsequent reaction.

In one or more embodiments, the gaseous stream is subject to aqueous scrubbing (e.g. aqueous spray) to capture and dissolve the catalyst (or residue thereof) within the gaseous stream. In these or other embodiments, the char is subjected to aqueous washing (e.g. float through an aqueous solution) to separate, through dissolution, the catalyst (or residue thereof). In these or other embodiments, the catalyst can be recovered from downstream dust collectors and then dissolved in an aqueous solution. In one or more embodiments, the aqueous solution used to dissolve the catalyst (or residue thereof) is adapted to facilitate dissolution of the catalyst. In one or more embodiments, the pH of aqueous solution is adjusted to target the solubility of the catalyst or residue thereof. For example, the pH of the aqueous solution can be adjusted to a pH of less than 6 in other embodiments less than 4, and in other embodiments less than 2. In other embodiments, the pH of the aqueous solution is adjusted to a pH of greater than 8, in other embodiments greater than 10, and in other embodiments greater than 12. The skilled person understands that the pH of the aqueous solution can be adjusted by introducing one or more mineral acids or bases.

Once the catalyst or residue thereof is dissolved in the aqueous solution, the catalyst or residue thereof can be precipitated, which may take place by neutralizing the solution, and then the precipitate can be recovered. As the skilled person will appreciate, the precipitate can be recovered by, for example, filter press or other forms of dewatering. The recovered catalyst can be dried prior to reintroducing the catalyst to the gasifier and/or the feed stock. In one or more embodiments, the catalyst is dried by using energy (e.g. heat) from the gasification reaction.

### Characteristics of Gaseous Product Stream

As indicated above, the gaseous product stream from the gasification reaction, which may be referred to as gasification product stream, gaseous stream, or syngas, includes advantageous levels of carbon monoxide relative to carbon dioxide. The product stream may also include advantageous levels of hydrogen. Still further, the product stream may include advantageous levels of methane.

In one or more embodiments, the gasification product stream includes greater than 10, in other embodiments greater than 20, and in other embodiments greater than 35 volume % carbon monoxide. In these or other embodiments, the gasification product stream includes less than 60, in other embodiments less than 50, and in other embodiments less than 45 volume % carbon monoxide. In one or more embodiments, the gasification product stream includes from about 10 to about 60, in other embodiments from about 20 to about 50, and in other embodiments from about 35 to about 45 volume % carbon monoxide.

In one or more embodiments, the gasification product stream includes greater than 5, in other embodiments greater than 10, and in other embodiments greater than 15 volume % carbon dioxide. In these or other embodiments, the gasification product stream includes less than 40, in other embodiments less than 30, and in other embodiments less than 25 volume % carbon dioxide. In one or more embodiments, the gasification product stream includes from about 5 to about 40, in other embodiments from about 10 to about 30, and in other embodiments from about 15 to about 25 volume % carbon dioxide.

In one or more embodiments, the gasification product stream includes greater than 5, in other embodiments greater than 10, and in other embodiments greater than 20 volume % hydrogen. In these or other embodiments, the gasification product stream includes less than 40, in other embodiments less than 35, and in other embodiments less than 30 volume % hydrogen. In one or more embodiments, the gasification product stream includes from about 5 to about 40, in other embodiments from about 10 to about 35, and in other embodiments from about 20 to about 30 volume % hydrogen.

### Conditioning Gasification Product Stream

In one or more embodiments, the gaseous stream is conditioned (i.e. treated) prior to further converting the gaseous stream (e.g. bioconversion). In one or more embodiments, the gaseous product stream from thermal decomposition may be pressurized. In one or more embodiments, pressurization of the gaseous stream achieves sufficient pressure to overcome counter forces within the bioreactor in which bioconversion may take place. As the skilled person understands, this will permit flow of the gas through the bioreactor and allow inert gases (e.g. nitrogen) within the gaseous stream to enter the head space of the reactor. In one or more embodiments, the gaseous stream is pressurized to a pressure of from about 5 to about 20 barr.

Also, the gaseous stream can be cooled. As the skilled person will appreciate, the gaseous stream can be cooled, for example, within a heat exchanger such as a water-cooled unit. In one or more embodiments, the gaseous stream is cooled to a temperature below that which would otherwise have a deleterious impact on the microorganism culture within the bioreactor. In one or more embodiments, the gaseous stream is cooled to a temperature of from about 25 to about 45° C. prior to delivery to the bioreactor.

Still further, the gaseous stream can be treated to remove undesirable constituents that may be entrained within the stream. For example, the gaseous stream can be treated within a scrubber prior to being introduced to the bioreactor. In one or more embodiments, this may include the use of a catalyst to remove hydrogen sulfide (e.g. an iron oxide catalyst). The stream may also be treated to remove halides (e.g. treatment with calcium or sodium carbonate).

### Syngas to Ethanol

In one or more embodiments, constituents of the gaseous stream are converted to ethanol (i.e. to produce an ethanol-containing stream). In one or more embodiments, the gaseous stream is supplemented with hydrogen prior to converting the gaseous stream to an ethanol-containing stream. Also, the gaseous stream can be treated to remove undesirable constituents prior to converting the gaseous stream to an ethanol-containing stream. For example, the skilled person understands that certain additives can be introduced to the gaseous stream to thereby remove (e.g. scavenge) sulfur and other impurities.

In one or more embodiments, constituents of the gaseous stream are converted to ethanol via biosynthetic techniques. For example, it is known that syngas can be converted to ethanol by fermentation utilizing microorganisms, such as bacteria. The microorganisms may be acetogenic autotrophic microbes. Acetogenic microbes (i.e. bacteria) generally transform the CO, H₂, and CO₂ in syngas into acetyl-CoA. The acetyl-CoA is then converted to organic products, such as acetic acid and ethanol. In one or more embodiments of the present invention, it may be desirable to preferentially produce ethanol relative to acetic acid.

As another potential pathway, some microorganisms (e.g. acetogens) can reduce the acetic acid (i.e. an organic acid) into an alcohol (e.g. ethanol). The acetogenic mechanism and the particular microorganisms chosen may be considered relative to preferentially producing ethanol. Microorganisms other than acetogens may be suitable with different pathways to reaching ethanol. The microorganisms may simultaneous uptake both CO and H₂ in the syngas.

The skilled person also appreciates that various conditions of microorganism-driven biosynthesis may be adjusted relative to preferential production of ethanol. For example, the pH, temperature, and concentrations of nutrients within the bioreactor where in the biosynthesis takes place can be adjusted. The desirable pH, temperature, and concentrations of nutrients may depend on the particular microorganisms employed.

According to embodiments of the invention, the resultant ethanol (i.e. from the bioreactor) is contained with an ethanol-containing stream. According to embodiments of the invention, a crude ethanol-containing stream, which is obtained directly from the bioreactor in which the ethanol is synthesized, is delivered to downstream steps. In other embodiments, the ethanol-containing stream from the bioreactor is purified to produce an ethanol-containing stream that is higher in ethanol content. In one or more embodiments, the ethanol-containing stream exiting the step wherein syngas is converted to ethanol (i.e. within the bioreactor) and delivered to the downstream steps where butanediol is converted (optionally after purification) includes greater than 80 wt %, in other embodiments greater than 90 wt %, and in other embodiments greater than 95 wt % ethanol based upon the entire weight of the ethanol-containing stream.

### Syngas to Butanediol

In one or more embodiments, constituents of the gaseous stream are converted to butanediol that is contained with a butanediol-containing stream; i.e. a product stream that includes butanediol. In one or more embodiments, the gaseous stream is supplemented with hydrogen prior to converting the gaseous stream to a butanediol-containing stream. Also, the gaseous stream can be treated to remove undesirable constituents prior to converting the gaseous stream to a butanediol-containing stream. For example, the skilled person understands that certain additives can be introduced to the gaseous stream to thereby remove (e.g. scavenge) sulfur and other impurities.

In one or more embodiments, the gaseous stream is converted to a butanediol-containing stream via biosynthetic techniques. For example, it is known that syngas can be converted to butanediol by microorganisms (i.e. a microbial catalyst). Exemplary microorganisms include C. autoethanogenum, C. ljungdahlii, and C. ragsdalei. These microorganisms generally utilize the Wood-Ljungdahl metabolic pathway to achieve the butanediol. Other microorganisms that produce butanediol by different pathways may also be suitable. Also, the skilled person understands that various parameters of biosynthetic process can be adjusted relative to preferential production of butanediol. For example, manipulation of pH, temperature, and concentrations of nutrients can lead to preferred production of the desired butanediol product. Also, the skilled person understands that the desirable pH, temperature, and concentrations of nutrients may depend on the particular microorganisms employed.

As indicated above, the resultant butanediol is contained with a butanediol-containing stream. According to embodiments of the invention, 1,3-butanediol is the targeted butanediol isomer; i.e., 1,3-butanediol is preferred relative to 2,3-butanediol. Advantageously, the use of the feedstock of the present invention leads to production of the favored 1,3-butanediol. In one or more embodiments, the butanediol within the butanediol-containing stream includes greater than 30, in other embodiments greater than 40, in other embodiments greater than 50, in other embodiments greater than 60, in other embodiments greater than 70, and in other embodiments greater than 80 wt %, of 1,3-butanediol based on the total weight of butanediol within the butanediol-containing stream.

In one or more embodiments, a crude butanediol-containing stream, which is obtained directly from the bioreactor in which the butanediol is synthesized, is delivered to downstream steps. In other embodiments, the butanediol-containing stream is purified to produce a butanediol-containing stream that is higher in butanediol content. In one or more embodiments, the butanediol-containing stream exiting the step wherein syngas is converted to butanediol and delivered to the downstream steps where butanediol is converted (optionally after purification) includes greater than 80 wt %, in other embodiments greater than 90 wt %, and in other embodiments greater than 95 wt % butanediol based upon the entire weight of the butanediol-containing stream.

### Syngas to Alkanes

In one or more embodiments, constituents of the gaseous stream are converted to alkanes that are contained within an alkane-containing stream. In one or more embodiments, the gaseous stream may be treated to remove undesirable constituents prior to converting the gaseous stream to an alkane-containing stream. For example, the skilled person understands that certain additive can be introduced to the gaseous stream to thereby remove (e.g. scavenge) sulfur and other impurities such as water and carbon dioxide.

In one or more embodiments, the gaseous stream is converted to alkanes by Fischer-Tropsch techniques. These techniques are generally known and include a variety of chemical reactions for converting a mixture of carbon monoxide and hydrogen into hydrocarbons (e.g. liquid hydrocarbons). As the skilled person appreciated, Fischer-Tropsch processes can be tailored to favor the production of certain hydrocarbons relative to other hydrocarbons with the alkane-containing stream. According to aspects of this invention, this may include tailoring the process to favor the production of n-butane, which can be subsequently dehydrogenated butadiene. In other embodiments, the Fischer-Tropsch process can be tailored to favor the production of naphtha including light naphtha (C4-C6 hydrocarbons) and/or heavy naphtha (C7-C10 hydrocarbons). In one or more embodiments, the Fischer-Tropsch techniques employed in practicing the present invention may be operated at a temperature of from about 150° C. to about 300° C. Useful catalysts conducting Fischer-Tropsch synthesis according to aspects this invention include the transition metals catalysts such as, but not limited to, those including cobalt, iron, ruthenium, and/or nickel.

As indicated above, the alkanes are contained within an alkane-containing stream. According to embodiments of the invention, a crude alkane-containing stream, which is obtained directly from the Fischer-Tropsch reactor(s) is delivered to downstream steps. In other embodiments, the alkane-containing stream is purified to produce a stream that is higher in alkane content and/or higher in the content of a particular alkane. In one or more embodiments, the alkane-containing stream exiting the Fischer-Tropsch reactor(s) is delivered to the downstream steps where butadiene is produced includes greater than 80 wt %, in other embodiments greater than 90 wt %, and in other embodiments greater than 95 wt % ethanol based upon the entire weight of the ethanol-containing stream. In these or other embodiments, the alkane-containing streams delivered to downstream processing include greater than 80 wt %, in other embodiments greater than 90 wt %, and in other embodiments greater than 95 wt % butane based upon the entire weight of the alkane-containing stream. In other embodiments, the alkane-containing streams delivered to downstream processing include greater than 80 wt %, in other embodiments greater than 90 wt %, and in other embodiments greater than 95 wt % light and/or heavy naphtha based upon the entire weight of the alkane-containing stream.

### Butadiene Synthesis

### Ethanol to Butadiene—One-Step Synthesis

In one or more embodiments, ethanol within the ethanol-containing stream can be converted to butadiene (e.g. 1,3-butadiene) by a one-step synthesis, which may also be referred to as a direct synthesis. In one or more embodiments, the direct synthesis of ethanol to butadiene takes place as a condensation reaction in the presence of a polyfunctional catalyst, including those disclosed in U.S. Pat. No. 8,921,635, which is incorporated herein by reference. Another known synthesis for converting ethanol directly to butadiene is the Lebedev process. Still other processes for directly converting ethanol to butadiene include those marketed by ETB Catalytic Technologies and those techniques marketed by Synthos.

As the skilled person understands, the direct conversion of ethanol to butadiene may include the use of a two-stage extractive distillation that utilizes n-methyl pyrrolidone (NMP) as solvent. Direct conversion techniques may also include conventional distillation for recovering the butadiene.

### Ethanol to Butadiene—Two-Step Synthesis

In alternative embodiments, ethanol within the ethanol-containing stream can be converted to butadiene (e.g. 1,3-butadiene) by a two-step synthesis, wherein acetaldehyde is synthesized as an intermediate. In one or more embodiments, the acetaldehyde, which is contained within an acetaldehyde-containing stream, is isolated before delivered to downstream synthesis steps. In other embodiments, the acetaldehyde-containing stream, as a crude stream, is delivered to downstream synthesis steps. According to embodiments of the invention, the acetaldehyde is reacted with ethanol in a second step to yield the desired 1,3-butadiene.

As the skilled person appreciates, ethanol can be converted to acetaldehyde by oxidative dehydrogenation of the ethanol. In one or more embodiments, the conversion of ethanol to acetaldehyde generally proceeds by partial oxidation of the ethanol in an exothermic reaction. In this partial oxidation process, the reaction may be conducted over a silver catalyst at about 500° C. to about 650° C. In one or more embodiments, the conversion of ethanol to acetaldehyde may include avoiding or inhibiting the production of acetic acid relative to the acetaldehyde. This can be accomplished by selecting catalysts and/or reaction conditions that are known to avoid the production of acetic acid relative to the acetaldehyde. In one or more embodiments, the conversion of ethanol to acetaldehyde takes place in the absence of evaporating the acetaldehyde. In one or more embodiments, the conversion of ethanol to acetaldehyde takes place at relatively low pressure and in the gas-phase as to increase selectivity for acetaldehyde. In one or more embodiments, the first step of partial dehydrogenation of ethanol to acetaldehyde to achieve an ethanol-acetaldehyde mixture, and a second step for converting the ethanol-acetaldehyde mixture to butadiene 200. This may include supplementing the ethanol-acetaldehyde mixture with additional ethanol and/or acetaldehyde as to achieve a desirable ratio. This process may be referred to as an Ostromislensky process.

### Alkanes to Butadiene

Alternatively, where syngas is converted to alkanes, alkanes within the alkane-containing stream can be treated or otherwise processed in downstream steps to produce butadiene. For example, naphtha (either light or heavy) can be treated within a steam cracking process. As the skilled person appreciates, steam cracking generally includes thermally cracking the hydrocarbons (e.g. naphtha) by employing steam in a steam cracking furnace. Steam cracking generally employs relatively higher temperatures (e.g. about 850° C.). As a result of steam cracking, lighter hydrocarbons are produced including butane, butene and butadiene. The skilled person knows that steam cracking can be operated to preferentially produce butadiene. This may include the selection of a particular catalyst and/or reactor design to preferentially produce butadiene. The butane and/or butene can then be dehydrogenated to produce butadiene.

### Butanediol to Butadiene

Alternatively, where syngas is converted to butanediol, butanediol (e.g. 1,3-butanediol) within the butanediol-containing stream is converted to butadiene (e.g. 1,3-butadiene) that is contained within a butadiene-containing stream. In one or more embodiments, this is accomplished by a dehydration reaction. Methods for the dehydration of butanediol to butadiene are well known. For example, useful methodologies are disclosed in U.S. Pat. No. 9,434,659, which is incorporated herein by reference.

The dehydration of butanediol to butadiene is a well-known reaction that the skilled person appreciates can be accomplished in the presence of a catalyst. Several catalyst are known for promoting this dehydration reaction including, but not limited to, medium-pore zeolite catalysts, rare-earth orthophosphate catalysts, alumina catalysts, a sodium polyphosphate catalysts, and aluminosilicate catalysts. The skilled person also appreciates that rare-earth orthophosphate catalysts and alumina catalysts are advantageously employed when 2,3-butanediol is the target substrate, and sodium polyphosphate catalysts and aluminosilicate catalysts are advantageously employed when 1,3-butanediol is the target substrate. Where aluminosilicate catalysts are employed, they may advantageously be adjusted in order to preferentially produce butadiene. For example, the SiO₂/Al₂O₃ ratio and pore architecture of the aluminosilicate catalyst may be adjusted and/or it may be desirable to utilize relatively low acid-site densities in the aluminosilicate catalyst.

In one or more embodiments, the dehydration of butanediol to butadiene takes place at temperatures of from about 200 to about 300° C., or in other embodiments from about 200 to about 270° C. In one or more embodiments, butanediol is dehydrated to butadiene in the gas phase.

### INDUSTRIAL APPLICABILITY

In one or more embodiments, the butadiene monomer (e.g. 1,3-butadiene) produced by the methods of this invention can be used in the production of polybutadiene or butadiene copolymers (which may also be referred to as polybutadiene copolymers). For purposes of this specification, polymers produced from butadiene made from the gasification of used tires may be referred to as circular synthetic rubber, or circular synthetic polybutadiene to butadiene copolymers. In one or more embodiments, this circular synthetic rubber can be used in the manufacture of tire components. As a result, practice of the present invention provides a method by which waste material, in particular waste material from used tires, is converted back to useful tires. In other words, a tire recycling or tire circularity method is provided.

The synthesis of polybutadiene or polybutadiene copolymers from butadiene monomer is well known and can be accomplished by using several synthetic routes (i.e. polymerization mechanisms and techniques). For example, the monomer can be polymerized by free-radical emulsion polymerization, anionic polymerization, or coordination catalysis using, for example, nickel or neodymium-based catalyst systems.

As the skilled person appreciates, comonomers that can be copolymerized with butadiene to form polybutadiene copolymers include, but are not limited to, vinyl aromatic monomer such as styrene, as well as other diene monomer such as isoprene. In one or more embodiments, the comonomer is a sustainable comonomer. For example, styrene can be obtained from bio-synthesized feedstock, such as bioethanol, that is subsequently converted to styrene; see, e.g. U.S. Pat. No. 9,663,445. Alternatively, styrene can be synthesized from bio-based materials such as cinnamic acid or hydrocinnamic acid; see, U.S. Pat. No. 9,868,853. Yet other examples include styrene obtained from the depolymerization of polystyrene from post-consumer waste; see U.S. Publication No. 2022/0411351. The skilled person also understands that styrene can be obtained from those processes that are mass balanced to qualify as bio-based, bio-circular, or circular as designated by the International Sustainability and Carbon Certification ISCC.

It should be appreciated that the polybutadiene polymers prepared by polymerizing the butadiene of the present invention have a relatively high percentage of mer units deriving from the butadiene produced by the invention, and therefore the polybutadiene polymers of this invention have a relatively high sustainable content. In one or more embodiments, the polybutadiene polymers synthesized by polymerizing the butadiene of the present invention include greater than 50 mol %, in other embodiments greater than 60 mol %, in other embodiments greater than 70 mol %, in other embodiments greater than 80 mol %, in other embodiments greater than 90 mol %, in other embodiments greater than 95 mol %, and in other embodiments greater than 99 mol % sustainable mer units, which obtained from polymerization of butadiene obtained by the present invention (i.e. mer units obtained from monomer that is synthesized from a gaseous stream obtained by gasification of carbonaceous materials). Likewise, where the polymer synthesized is a polybutadiene copolymer by copolymerizing the butadiene monomer obtained by practice of this invention together with sustainable comonomer, the resulting polybutadiene copolymer has a relatively high sustainable content. In one or more embodiments, the polybutadiene copolymers synthesized by polymerizing the butadiene of the present invention together with sustainable comonomer include greater than 50 mol %, in other embodiments greater than 60 mol %, in other embodiments greater than 70 mol %, in other embodiments greater than 80 mol %, in other embodiments greater than 90 mol %, in other embodiments greater than 95 mol %, and in other embodiments greater than 99 mol % sustainable mer units (i.e. mer units that are obtained from monomer synthesized from a gaseous stream obtained by gasification of carbonaceous materials and other sustainable comonomer).

The polymers synthesized from butadiene monomer produced by embodiments of the invention may be referred to as vulcanizable polymers, or as elastomeric polymers, and generally include polydienes and polydiene copolymers. Specific of polymers that can be produced and used in the manufacture of tires include, but are not limited to, polybutadiene, poly(styrene-co-butadiene), poly(styrene-co-isoprene-co-butadiene), poly(isoprene-co-butadiene), and functionalized derivatives thereof.

The polybutadiene and polybutadiene copolymers produced by the present invention exhibit excellent viscoelastic properties and are particularly useful in the manufacture of various tire components including, but not limited to, tire treads, sidewalls, subtreads, and bead fillers. These polymers can be used as all or part of the elastomeric component of a tire stock. When the polymers produced by the present invention are used in conjunction with other vulcanizable polymers to form the elastomeric component of a tire stock, these other vulcanizable polymers may include natural rubber, synthetic rubbers, and mixtures thereof. Examples of synthetic rubber include polyisoprene, poly(styrene-co-butadiene), and other polybutadienes with low and/or cis-1,4-linkage content, poly(styrene-co-butadiene-co-isoprene), and mixtures thereof. The polymers of this invention can also be used in the manufacture of hoses, belts, shoe soles, window seals, other seals, vibration damping rubber, and other industrial products.

Practice of the present invention not only offers a method for recycling tires by employing used tires as a feedstock to produce polymer that can be formulated back into tires, but the practice of the present invention also advantageously provides a method whereby a tire is produced that has a relatively high content of sustainable constituents, which include recycled materials, naturally-derived materials and/or materials synthesized from bio-synthesized feedstock or bio-based materials. Moreover, these tires or tire components include threshold amounts of circular synthetic rubber while being characterized by high sustainable content. For example, the tires or tire components of the present invention can include greater than 40 wt %, in other embodiments greater than 50 wt %, and in other embodiments greater than 60 wt % sustainable materials. In these or other embodiments, the tire or tire components include from about 40 to about 90 wt %, in other embodiments from about 45 to about 85 wt %, and in other embodiments from about 50 to about 80 wt % sustainable material. In combination therewith, the rubber component of the tires or tire components of the present invention include greater than 10 wt %, in other embodiments greater than 20 wt %, in other embodiments greater than 30 wt %, in other embodiments greater than 40 wt %, in other embodiments greater than 45 wt %, and in other embodiments greater than 50 wt % circular synthetic rubber, which includes synthetic rubber produced according to embodiments of the present invention.

In one or more embodiments, the present invention provides vulcanizable compositions including a rubber component that is a circular synthetic rubber produced according to aspects of this invention. The rubber component may also include other synthetic rubber, such as synthetic rubber that derives from petroleum-based raw materials and has not been recycled, synthetic rubber that derives from other sustainable processes, as well as natural rubber. As the skilled person understands, natural rubber is synthesized by and obtained from plant life. For example, natural rubber can be obtained from Hevea rubber trees, guayule shrub, gopher plant, mariola, rabbitbrush, milkweeds, goldenrods, pale Indian plantain, rubber vine, Russian dandelions, mountain mint, American germander, and tall bellflower.

Other synthetic polymers, if used, can include, without limitation, synthetic polyisoprene, polybutadiene, polyisobutylene-co-isoprene, neoprene, poly(ethylene-co-propylene), poly(styrene-co-butadiene), poly(styrene-co-isoprene), poly(styrene-co-isoprene-co-butadiene), poly(isoprene-co-butadiene), poly(ethylene-co-propylene-co-diene), polysulfide rubber, acrylic rubber, urethane rubber, silicone rubber, epichlorohydrin rubber, and mixtures thereof. These elastomers can have a myriad of macromolecular structures including linear, branched, and star-shaped structures.

Generally, the rubber compositions of this invention include from about 30 to about 65 wt %, in other embodiments from about 35 to about 60 wt %, and in other embodiments from about 40 to about 55 wt % elastomer, based on the total weight of the tire component.

As suggested above, the rubber compositions include fillers such as organic and inorganic fillers. Examples of organic fillers include carbon black and starch. Examples of inorganic fillers include silica, aluminum hydroxide, magnesium hydroxide, mica, talc (hydrated magnesium silicate), and clays (hydrated aluminum silicates). In certain embodiments, a mixture of different fillers may be advantageously employed.

The amount of total filler employed in the rubber compositions can be up to about 150 parts by weight per 100 parts by weight of rubber (phr), with about 30 to about 125 phr, or about 40 to about 110 phr being typical. In certain embodiments the total filler content is greater than about 100 phr. In other embodiments, the total filler content is from about 50 to about 100 phr, and in in further embodiments from about 55 to about 95 phr.

Conventional carbon black can be used, which is generally known in the art. In one or more embodiments, carbon blacks include furnace blacks, channel blacks, and lamp blacks. More specific examples of carbon blacks include super abrasion furnace blacks, intermediate super abrasion furnace blacks, high abrasion furnace blacks, fast extrusion furnace blacks, fine furnace blacks, semi-reinforcing furnace blacks, medium processing channel blacks, hard processing channel blacks, conducting channel blacks, and acetylene blacks.

In particular embodiments, the carbon blacks may have a surface area (EMSA) of at least 20 m²/g and in other embodiments at least 35 m²/g; surface area values can be determined by ASTM D-1765 using the cetyltrimethylammonium bromide (CTAB) technique. The carbon blacks may be in a pelletized form or an unpelletized flocculent form. The preferred form of carbon black may depend upon the type of mixing equipment used to mix the rubber compound.

In one or more embodiments, carbon black can be sourced from a recycled material. Such recycled material can include reclaimed or recycled vulcanized rubber, whereby the vulcanized rubber is typically reclaimed from manufactured articles such as a pneumatic tire, an industrial conveyor belt, a power transmission belt, and a rubber hose. The recycled carbon black may be obtained by a pyrolysis process or other methods known for obtaining recycled carbon black. In an aspect, a recycled carbon black can be formed from incomplete combustion of recycled rubber feedstock or rubber articles. In another aspect, the recycled carbon black can be formed from the incomplete combustion of feedstock including oil resulting from the tire pyrolysis process. The carbon blacks utilized in the preparation of the vulcanizable elastomeric compositions can be in pelletized form or an unpelletized flocculent mass.

The amount of carbon black employed in the rubber compositions can be up to about 75 parts by weight per 100 parts by weight of rubber (phr), with about 5 to about 60 phr, or about 10 to about 55 phr being typical.

The rubber composition can further include filler in the form of one or more recycled rubbers in a particulate form. Recycled particulate rubber is typically broken down and reclaimed (or recycled) by any of a plurality of processes, which can include physical breakdown, grinding, chemical breakdown, devulcanization, cryogenic grinding, a combination thereof, etc. The term recycled particulate rubber can relate to both vulcanized and devulcanized rubber, where devulcanized recycle or recycled rubber (reclaim rubber) relates to rubber which has been vulcanized, ground into particulates and may have further undergone substantial or partial devulcanization. In an example, the recycled particulate rubber used in the rubber composition is essentially free of recycled rubber resulting from devulcanization. In a situation where the vulcanized rubber contains wire or textile fiber reinforcement, such wire or fiber reinforcement can be removed by any suitable process such as magnetic separation, air aspiration and/or air flotation step. In certain embodiments, the “recycled particulate rubber” comprises cured, i.e., vulcanized (crosslinked) rubber that has been ground or pulverized into particulate matter having a mean average particle size as discussed below.

Certain silicas may be considered sustainable materials. Some commercially available silicas which may be used as sustainable materials for the current invention include Hi-Sil™ 215, Hi-Sil™ 233, and Hi-Sil™ 190 (PPG Industries, Inc.; Pittsburgh, Pa.). Other suppliers of commercially available silica include Grace Davison (Baltimore, Md.), Degussa Corp. (Parsippany, N.J.), Rhodia Silica Systems (Cranbury, N.J.), and J.M. Huber Corp. (Edison, N.J.). Other sustainable silicas include those derived from rice husk ash.

In one or more embodiments, silicas may be characterized by their surface areas, which give a measure of their reinforcing character. The Brunauer, Emmet and Teller (“BET”) method (described in J. Am. Chem. Soc., 1939, vol. 60, 2 p. 309-319) is a recognized method for determining the surface area. The BET surface area of silica is generally less than 450 m²/g. Useful ranges of surface area include from about 32 to about 400 m²/g, about 100 to about 250 m²/g, and about 130 to about 240 m²/g, and about 170 to about 220 m²/g. In certain embodiments, the silica may have a BET surface area of 190 to about 280 m²/g. The pH's of the silicas are generally from about 5 to about 7 or slightly over 7, or in other embodiments from about 5.5 to about 6.8.

In one or more embodiments, where silica is employed as a filler (alone or in combination with other fillers), a coupling agent and/or a shielding agent may be added to the rubber compositions during mixing in order to enhance the interaction of silica with the elastomers. Useful coupling agents and shielding agents are disclosed in U.S. Pat. Nos. 3,842,111; 3,873,489; 3,978,103; 3,997,581; 4,002,594; 5,580,919; 5,583,245; 5,663,396; 5,674,932; 5,684,171; 5,684,172; 5,696,197; 6,608,145; 6,667,362; 6,579,949; 6,590,017; 6,525,118; 6,342,552; and 6,683,135; which are incorporated herein by reference.

The amount of silica employed in the rubber compositions can be from about 1 to about 150 phr or in other embodiments from about 5 to about 130 phr. The useful upper range is limited by the high viscosity imparted by silicas. In certain embodiments, the silica employed in the rubber composition is derived from rice husk ash only, and in other embodiments the rubber compositions do not include silica from non-rice husk ash derived processes. When silica is used together with carbon black, the amount of the silica or carbon black individually can be as low as about 1 phr. Generally, the amounts of coupling agents and shielding agents range from about 4 wt % to about 20 wt % based on the weight of silica used. In one or more embodiments, where carbon black and silica are employed in combination as a filler, the weight ratio or silica to total filler may be from about 5 wt % to about 99 wt % of the total filler, in other embodiments from about 10 wt % to about 90 wt % of the total filler, or in yet other embodiments from about 50 wt % to about 85 wt % of the total filler. In certain embodiments the silica and carbon black fillers employed in the rubber composition are selected from the group consisting of sustainable pyrolysis carbon black and/or rice husk ash derived silica.

A multitude of rubber curing agents (also called vulcanizing agents) may be employed, including sulfur or peroxide-based curing systems. Curing agents are described in Kirk-Othmer, ENCYCLOPEDIA OF CHEMICAL TECHNOLOGY, Vol. 20, pgs. 365-468, (3rd Ed. 1982), particularly Vulcanization Agents and Auxiliary Materials, pgs. 390-402, and A. Y. Coran, Vulcanization, ENCYCLOPEDIA OF POLYMER SCIENCE AND ENGINEERING, (2nd Ed. 1989), which are incorporated herein by reference. Vulcanizing agents may be used alone or in combination.

Other ingredients that are typically employed in rubber compounding may also be added to the rubber compositions. These include accelerators, accelerator activators, oils, plasticizer, waxes, scorch inhibiting agents, processing aids, zinc oxide, tackifying resins, reinforcing resins, fatty acids such as stearic acid, peptizers, and antidegradants such as antioxidants and antiozonants.

With regard to oils, sustainable oils, which include plant-based oils and bio-based oils, may be used. Plant-based oils may include plant-based triglycerides. Exemplary oils include, without limitation, palm oil, soybean oil (also referred to herein as soy oil), rapeseed oil, sunflower seed, peanut oil, cottonseed oil, oil produced from palm kernel, coconut oil, olive oil, corn oil, grape seed oil, hemp oil, linseed oil, rice oil, safflower oil, sesame oil, mustard oil, flax oil. Other examples include nut-derived oils such oils obtained from beech nuts, cashews, mongongo nuts, macadamia nuts, pine nuts, hazelnuts, chestnuts, acorns, almonds, pecans, pistachios, walnuts, or brazil nuts. As the skilled person will appreciate, these oils can be produced by any suitable process such as mechanical extraction (e.g., using an oil mill), chemical extraction (e.g., using a solvent, such as hexane or carbon dioxide), pressure extraction, distillation, leaching, maceration, purification, refining, hydrogenation, sparging, etc.

Bio-based oils, also referred to as bio-oils, can include oils produced by a recombinant cell. For example, bio-oils produced by recombinant cells can be produced using a select strain of algal cells that are fed with a supply of sugars (e.g., sucrose) and then allowed to ferment and produce a bio-oil with a selected profile; after sufficient growth or fermentation has taken place, the bio-oil is isolated from the cells and collected.

Generally, the rubber compositions of this invention can include from about 1 to about 70 parts by weight, or in other embodiments from about 5 to about 50 parts weight total oil per 100 parts by weight rubber. The amount of sustainable oil, relative to the total weight of oil included, may be from about 1 wt % to about 99 wt %, or in other embodiment from about 20 wt % to about 80 wt %.

With regard to waxes, the rubber compositions can include one or more sustainable waxes, which include natural waxes. A natural wax, or one with no petroleum as its raw material, can include carnauba wax, candelilla wax (e.g., extracted from candelilla flowers), rice wax (e.g., separated from rice bran oil) and Japan wax (e.g., extracted from Japanese wax tree).

Generally, the rubber compositions of this invention include from about 1 to about 20 parts by weight, or in other embodiments from about 2 to about 15 parts by weight total wax per 100 parts by weight rubber. The amount of sustainable wax, relative to the total weight of wax included, may be from about 1 wt % to about 99 wt %, or in other embodiment from about 20 wt % to about 80 wt % of the total wax. In certain embodiments, the rubber composition includes sustainable waxes only.

All ingredients of the rubber compositions can be mixed with standard mixing equipment such as Banbury or Brabender mixers, extruders, kneaders, and two-rolled mills. In one or more embodiments, the ingredients are mixed in two or more stages. In the first stage (often referred to as the masterbatch mixing stage), a so-called masterbatch, which typically includes the rubber component and filler, is prepared. To prevent premature vulcanization (also known as scorch), the masterbatch may exclude vulcanizing agents. The masterbatch may be mixed at a starting temperature of from about 25° C. to about 125° C. with a discharge temperature of about 135° C. to about 180° C. Once the masterbatch is prepared, the vulcanizing agents may be introduced and mixed into the masterbatch in a final mixing stage, which is typically conducted at relatively low temperatures so as to reduce the chances of premature vulcanization. Optionally, additional mixing stages, sometimes called remills, can be employed between the masterbatch mixing stage and the final mixing stage. One or more remill stages are often employed where the rubber composition includes silica as the filler. Various ingredients including the polymers of this invention can be added during these remills.

The mixing procedures and conditions particularly applicable to silica-filled tire formulations are described in U.S. Pat. Nos. 5,227,425; 5,719,207; and 5,717,022, as well as European Patent No. 890,606, all of which are incorporated herein by reference. In one embodiment, the initial masterbatch is prepared by including the polymer and silica in the substantial absence of coupling agents and shielding agents.

In order to fabricate tire components with the polymers produced by the invention, the skilled person appreciates that the polymers are mixed with various other ingredients (e.g. filler and curative) to produce a rubber composition (also referred to as vulcanizable composition), and the vulcanizable composition is then processed into tire components according to ordinary tire manufacturing techniques, which generally include standard rubber shaping and molding techniques. The tire components may include, but are not limited to, tire treads, sidewalls, subtreads, body ply skims, and bead filler. The various tire components are then assembled into a green tire (i.e. an uncured tired), placed within a mold, and then vulcanized. Typically, vulcanization is effected by heating the vulcanizable composition in a mold; e.g., it may be heated to about 140° C. to about 180° C. Cured or crosslinked rubber compositions may be referred to as vulcanizates, which generally contain three-dimensional polymeric networks that are thermoset. The other ingredients, such as fillers and processing aids, may be evenly dispersed throughout the crosslinked network. Pneumatic tires can be made as discussed in U.S. Pat. Nos. 5,866,171; 5,876,527; 5,931,211; and 5,971,046, which are incorporated herein by reference.

In one or more embodiments, the tires can include fabric reinforcement made by using non-petroleum materials in place of synthetic fibers. For example, mechanical recycled fibers, chemical recycled fibers, or bio-based fibers can be used. Likewise, the tires can include metal reinforcement made from recycled steel and/or other circular or sustainable metals. These non-petroleum fabrics and recycled metals can be used exclusively within the tires or in combination with traditional fabric and/or metal reinforcement.

Various modifications and alterations that do not depart from the scope and spirit of this invention will become apparent to those skilled in the art. This invention is not to be duly limited to the illustrative embodiments set forth herein.

What is claimed is:

1. A gasification process comprising:
(i) providing a carbonaceous feedstock; and
(ii) gasifying the carbonaceous feedstock in the presence of a catalyst.

2. The gasification process of claim 1, where said step of gasifying takes place at a temperature of from about 500 to about 2500° C.

3. The gasification process of claim 2, where said step of gasifying takes place in the presence of an amount of oxygen to provide with an oxygen equivalence ratio of from about 0.05 to about 0.8.

4. The gasification process of claim 1, where catalyst is selected from metals, metal oxides, and metal salts of alkali metals, alkaline earth metals, and transition metals.

5. The gasification process of claim 1, where said step of gasifying takes place in the presence of from about 0.01 to about 20 wt % catalyst based upon weight of the feedstock.

6. The gasification process of claim 1, where the process includes applying the catalyst to the carbonaceous feedstock in the form of catalyst particles.

7. The gasification process of claim 6, where the catalyst particles are characterized by a D90 of from about 5 μm to about 5 mm.

8. The gasification process of claim 1, where the process includes applying the catalyst to the carbonaceous feedstock in the form of an emulsion or dispersion.

9. The gasification process of claim 1, where the feedstock includes used tires.

10. The gasification process of claim 1, where the feedstock includes municipal solid waste.

11. The gasification process of claim 1, where the feedstock includes biomass.

12. The gasification process of claim 1, where said step of gasifying produces a gaseous stream including (i) from about 10 to about 60 volume % carbon monoxide, (ii) produces a gaseous stream including from about 5 to about 40 volume % carbon dioxide, and (iii) produces a gaseous stream including from about 5 to about 40 volume % hydrogen.

13. The gasification process of claim 1, where constituents of the gaseous stream are biosynthetically converted to ethanol.

14. The gasification process of claim 1, where constituents of the gaseous stream are biosynthetically converted to butanediol.

15. The gasification process of claim 1, where constituents of the gaseous stream are biosynthetically converted to alkanes.

16. The gasification process of claim 1, where the constituents of the gaseous stream are biosynthetically converted to ethanol, butanediol and/or alkanes, and where the ethanol, butanediol and/or alkanes are converted to butadiene.

17. The gasification process of claim 16, where the butadiene is synthesized to polybutadiene.

18. The gasification process of claim 17, where polybutadiene is included within a tire.

19. The gasification process of claim 1, further comprising at least one of (i) the step recovering a portion of the catalyst or residue thereof from the gaseous stream, and (ii) where said step of gasifying produces a by-product char, and further comprising the step of recovering a portion of the catalyst or residue thereof from the char.

20. The gasification process of claim 19, where the catalyst or residue thereof recovered from the gaseous stream or the char is returned to the process.

21. The gasification process of claim 20, where greater than 30 wt % of the catalyst included in the gasification process is recovered and returned to the process.
